(19) United States
(12) Patent Application Publication
(10) Pub. No.: US 2013/0225498 A1
(43) Pub. Date: Aug. 29, 2013

(54) COMPOSITION FOR PREVENTING OR TREATING NEURODEGENERATIVE DISEASES CONTAINING CCL5

(71) Applicant: Kyungpook National University Industry-Academic, (US)

(72) Inventors: Jae-Sung BAE, Daegu (KR); Hee Kyung Jin, Daegu (KR); Jong Kil Lee, Daegu (KR)

(73) Assignee: Kyungpook National University Industry-Academic Cooperation Foundation, Daegu (KR)

(21) Appl. No.: 13/656,988

(22) Filed: Oct. 22, 2012

(30) Foreign Application Priority Data
Feb. 29, 2012 (KR) 10-2012-0021120

(51) Int. Cl. A61K 38/19; A61K 38/17

(52) U.S. Cl. CPC A61K 38/195; A61K 38/1716
USPC 514/17.8; 514/18.2

(57) ABSTRACT

There is provided a composition for preventing or treating neurodegenerative diseases, including one or two or more selected from the group consisting of a chemoattractant CCL5, a CCL5 expression regulator, and a CCL5 activator as an active ingredient, in which by confirming through a morris water maze task that the CCL5 recovers memory loss and improves spatial cognition ability, it has been found that the composition including any one or two or more selected from the group consisting of the CCL5, the CCL5 expression regulator, and the CCL5 activator can be usefully employed as a pharmaceutical composition or a food composition for preventing or treating neurodegenerative diseases.

This application claims the priority of Korean Patent Application No. 10-2012-0021120 filed on 29 Feb. 2012, in the Korean Intellectual Property Office, the disclosure of which is incorporated herein by reference.

### BACKGROUND OF THE INVENTION

1. Field of the Invention

The present invention relates to a composition for preventing or treating neurodegenerative diseases.

2. Description of the Related Art

In general, neurodegenerative diseases are primarily caused by a death of a brain cell, and include various diseases such as dementia, Parkinson's disease, a stroke, Huntington's disease, and Alzheimer's disease. In addition, an increase of neurodegenerative diseases such as senile dementia due to a sharp increase in an aged population has become a serious social problem in the modern world. However, effective medicines or treatments for preventing and treating such a disease are not yet developed up to now.

Alzheimer's disease that is one of major causes of dementia is likely to terribly occur in the aged and around 50% to 60% of people suffered from Alzheimer' s disease progresses to dementia. Alzheimer's disease is classified into a disease that consistently decreases cognitive ability. The pathophysiology of Alzheimer's disease is complex and such a disease is caused by several different biochemical routes. Among them, a disorder of β-amyloid protein metabolism, or a disorder of neurotransmission of glutamaterigic, adrenergic, serotonergic, or dopaminergic neuron have been mentioned as a cause for Alzheimer's disease. Above this, an inflammation, oxidation, and a hormone route became known as a cause for Alzheimer's disease. An ultimate goal of healing Alzheimer's disease is to make a full recovery by eliminating the disease itself and also to reduce and eliminate a cognitive disorder, a mental disorder, an abnormal behavior, and the like that are caused by dementia.

Many drugs are now believed to be used for treating Alzheimer's disease, but most of them are still under examination of medicinal effects. Furthermore, all of the medicines so far including the medicines that are being developed until now are prepared in order to slightly slow down the progress of Alzheimer's disease or to treat symptoms that are caused by Alzheimer's disease. Therefore; there are no medicines that are designed and prepared in order to fundamentally treat Alzheimer's disease itself,

A major target for developing a medicine for treating Alzheimer's disease is a disorder of neurotransmitter so far. There are only cholinesterase inhibitors (Aricept, Exelon, Reminyl, and the like) that target cholinergic neuron and Memantine that is a glutamate antagonist, which are now commercially available by obtaining FDA's approval. However, the medicines temporarily relieve the symptom only. Accordingly, a technique for developing a medicine for essentially treating a disease or suppressing the progress of the disease itself is acutely required.

### SUMMARY OF THE INVENTION

An aspect of the present invention provides a composition for preventing or treating neurodegenerative diseases, including any one or two or more selected from the group consisting of a chemoattractant CCL5, a CCL5 expression regulator, and a CCL5 activator as an active ingredient.

An aspect of the present invention also provides a pharmaceutical composition for preventing or treating neurodegenerative diseases, including any one or two or more selected from the group consisting of a chemoattractant CCL5, a CCL5 expression regulator, and a CCL5 activator as an active ingredient.

An another aspect of the present invention provides a health food composition for preventing or treating neurodegenerative diseases, including any one or two or more selected from the group consisting of a chemoattractant CCL5, a CCL5 expression regulator, and a CCL5 activator as an active ingredient.

According to an aspect of the present invention, there is provided a composition for preventing or treating neurodegenerative diseases, including any one or two or more selected from the group consisting of a chemoattractant CCL5, a CCL5 expression regulator, and a CCL5 activator as an active ingredient.

The neurodegenerative diseases may be any one or more diseases selected from a stroke, palsy, memory loss, memory damage, dementia, amnesia, Parkinson's disease, Alzheimer's disease, Pick's disease, Creutzfeld-Kacob disease, Huntington's disease, and Lou Gehrig's disease.

According to another aspect of the present invention, there is provided a pharmaceutical composition for preventing or treating neurodegenerative diseases, including any one or two or more selected from the group consisting of a chemoattractant CCL5, a CCL5 expression regulator, and a CCL5 activator as an active ingredient.

The pharmaceutical composition includes 0.1 parts to 50.0 parts by weight of any one or two or more selected from the group consisting of a CCL5, a CCL5 expression regulator, and a CCL5 activator, relative to 100 parts by weight of the total pharmaceutical composition.

According to another aspect of the present invention, there is provided a food composition for preventing or treating neurodegenerative diseases, including any one or two or more selected from the group consisting of a chemoattractant CCL5, a CCL5 expression regulator, and a CCL5 activator as an active ingredient.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 illustrates that Aβ-stimulated BM-MSC CM induces the migration of microglia in vitro. FIG. 1a illustrates an experimental design for the cell migration assay, FIG. 1b illustrates a migration of microglia upon exposure to BM-MSC CM with or without. Aβ treatment, FIG. 1c illustrates a dose-dependent increase of cell migration using Aβ-stimulated BM-MSC CM, and FIG. 1d illustrates a formation of actin stress fibers in microglia after a stimulation with a control media and Aβ treated BM-MSC CM [Control (control media from non-cultured BM-MSC), Aβ alone (10 μM aggregated Aβ alone treatment in control media), BM-MSC CM (CM from non Aβ-treated BM-MSC), and Aβ stimulated BM-MSC CM (CM from Aβ-treated BM-MSC)].

FIG. 2 illustrates cytokine expression profiles of BM-MSC CM after Aβ exposure in vitro, FIG. 2a illustrates a cytokine array of CM derived from BM-MSCs with or without Aβ stimulation, FIG. 2b illustrates a results of determining whether the increased cytokine levels in the CM were elevated in the cells depending on Aβ stimulation, FIG. 2c illustrates a result of confirming increased CCL5 levels by ELISA of the media from BM-MSCs with or without Aβ stimulation [Control (control media from non-cultured BM-MSC), Aβ (10 μM aggregated Aβ alone treatment in control media), BM-MSC CM (CM from non Aβ-treated BM-MSC), and Aβ stimulated BM-MSC CM (CM from Aβ-treated BM-MSC)], and FIG. 2d illustrates a result of confirming whether BV2 microglia was migrated by recombinant murine CCL5 as a chemoattractant.

FIG. 3 illustrates that soluble CCL5 derived from BM-MSCs and activated by Aβ is a critical factor that, promotes BV2 microglia (FIG. 3a) and primary cultured microglia (FIG. 3b) migrations in vitro [Control (control media from non-cultured BM-MSC), BM-MSC CM (CM from non Aβ-treated BM-MSC), CCL5 siRNA BM-MSC CM (CM from non Aβ-treated CCL5 siRNA BM-MSC), Aβ stimulated BM-MSC CM (CM from Aβ-treated BM-MSC), and Aβ stimulated CCL5 siRNA BM-MSC CM (CM from Aβ-treated CCL5 siRNA BM-MSC)], and FIG. 3c illustrates CCL5 mRNA and protein level and after stimulating BM-MSCs treated with CCL5 siRNA with Aβ when compared with BM-MSCs treated with control siRNA.

FIG. 4 illustrates that CCL5 derived from BM-MSCs following transplantation into the Alzheimer's disease mouse (APP/PS1) brain is a critical factor to recruit BM-derived microglia. FIG. 4a illustrates a timeline of the experiment, FIG. 4b illustrates a result of determining whether BM-MSCs elevated CCL5 secretion in the mouse brains (cortex and hippocampus), FIG. 4c illustrates representative immunofluorescence images of microglia in the hippocampus of APP/PS1 mice using Iba-1 antibody, FIG. 4d illustrate the total numbers of GFP positive cells in the AD-GFP chimeric brain that were treated with PBSf BM-MSCs or CCL5 knockdown BM-MSCs (n=3 per group, scale bar, 50 μm), FIG. 4e illustrates a flow cytometry analysis showed that GFP+/CD45^{dim} microglia and GFP+/CB45^{high} macrophage increased in the BM-MSC treated chimeric AD group 3 days after the last treatment compared with the PBS group, and FIG. 4f illustrates brain sections of AD-GFP chimeric/BM-MSCs mice were analyzed 14 days after the last BM-MSC transplantation by confocal microscopy.

FIG. 5 illustrates that CCL5 derived from transplanted BM-MSCs modulates the microglial activation status in APP/PS1 mice. FIG. 5a and 5b illustrate that at 3 days after the last BM-MSC injection, the mRMA expression levels of immune-associated cytokines were measured by quantitative real-time PGR, FIG. 5c illustrates an evaluation of the TNF-α and IL-4 protein content in the hippocampus by ELISA, and FIG. 5d illustrates triple immunofluorescent images from BM-MSC treated AD-GFP chimeric mice showed that, the BM-derived microglia (green plus blue) expressed IL-4.

FIG. 6 illustrates that BM-derived cells recruited by CCL5 released from transplanted BM-MSCs reduces Aβ deposition by expressing Aβ-degrading enzymes in APP/PS1 mouse brains. FIG. 6a illustrates brain sections that were stained with 6E10 antibody to detect Aβ after PBS, BM-MSC or CCL5 knockdown BM-MSC treatment, FIG. 6b illustrates the relative area occupied and numbers of Aβ plaques that were determined by unbiased stereology in the hippocampus of APP/PS1 mice, FIG. 6c illustrates coronal brain sections that were immunostained with anti-20G10 and anti-G30 (n=4 for each group) and aggregated Aβ□40 and 42 that were quantified by the plaque area of Aβ□immunoreactivity, FIG. 6d illustrates Aβ 40 and 42 in the brain hippocampus of APP/PS1 mice that were assessed by ELISA (n=4 for each group), FIG. 6e illustrates Aβ deposits and BM-derived cells that were immunostained using anti-Iba1 antibody on coronal sections of AD-GFP chimeric mice treated with PBS, BM-MSCs or CCL5 knockdown BM-MSCs, FIG. 6f illustrates the expression of IDE, NEP, and MMP9, enzymes related to degradation of Aβ, that was measured in the hippocampal regions with quantitative real-time RT-PCR (n=4 per group), FIG. 6g illustrates the levels of NEP expression in the brain hippocampal tissues that were measured by western blot analysis, and FIG. 6h illustrates immunofluorescent images from BM-MSC treated AD-GFP chimeric mice showed that the BM-derived cells expressed NEP.

FIG. 7 illustrates that CCL5 released following BM-MSC transplantation into Aβ-deposited brain improves behavioral abnormalities of APP/PS1 mice.

FIG. 7a illustrates escape latencies of APP/PS1 mice whose brains that were treated with PBS, BM-MSCs, or CCL5 knockdown BM-MSCs and WT mice over 10 days, FIG. 7b illustrates that swimming traces of each group performing the MWM task on day 10, FIG. 7c illustrates the number of times the mice crossed platform area, and FIG. 7d illustrate that spent time in the target quadrant, that were measured during the 60 s.

FIG. 8 is a schematic illustration of the therapeutic, effects obtained in the APP/PS1 AD mouse model after intracerebral BM-MSC transplantation.

### DETAILED DESCRIPTION OF THE PREFERRED EMBODIMENT

The present invention provides a composition, especially, a pharmaceutical composition, and a health food composition, for preventing or treating neurodegenerative diseases, including any one or two or more selected from the group consisting of a CCL5, a CCL5 expression regulator, and a CCL5 activator as an active ingredient.

Hereinafter, the present invention will be described in detail.

The present inventors confirmed that neurodegenerative diseases can be treated through transplanting bone marrow mesenchymal stem cells (BM-MSCs) to brain suffered from neurodegenerative diseases; mRNA of a CCL5 among chemotaxis cytokines, which are up-regulated in bone marrow mesenchymal stem cells after amyloid beta (Aβ) stimulation, are significantly increased in mesenchymal stem cells and a damaged memory is recovered in the brain expressed with the CCL5 during trying to find a factor that is crucial for treating neurodegenerative diseases; and thus the CCL5 is an active ingredient for preventing or treating neurodegenerative diseases. The present inventors thus completed the present invention.

The present invention provides a composition for preventing or treating neurodegenerative disease, including any one or two more selected from the group consisting of a chemoattractant CCL5, a CCL5 expression regulator, and a CCL5 activator as an active ingredient.

A CCL5 is an 8 kDa protein that is classified into chemotaxis cytokines or chemokines, and also known to as RANTES (Regulated upon Activation, Normal T-cell Expressed, and Secreted). The chemotactic activity of the CCL5 induce the recruitment of T-cell, dendritic cells, NK cells, granulocytes, macrophases, or monocytes to an inflammation region singly or by stimulating MCP-1 production.

Such the CCL5 is released by bone marrow mesenchymal stem cells that are exposed with Aβ stimulation, but the present invention is not limited thereto. In addition, the range of the present invention is not limited to a route of obtaining, such as the production by genetic engineering technology, the production by chemical synthetic method, and the production by extracting from animals.

A type of such neurodegenerative diseases may include any one or more diseases selected from the group consisting of a stroke, palsy, memory loss, memory damage, dementia, amnesia, Parkinson's disease, Alzheimer's disease. Pick's disease, Creutzfeid-Kacob disease, Huntington's disease, and Lou Gehrig's disease, but the present invention is not particularly limited thereto. Preferably, a type of such neurodegenerative diseases may be any one or more diseases selected from the group consisting of memory loss, memory damage, dementia, amnesia, and Alzheimer's disease.

As confirmed in one embodiment of the present invention, it has been found that in the case of transplanting bone marrow mescenchymal stem cells that are deposited with Aβ, a CCL5 released from bone marrow mescenchymal stem cells allows memory loss to be recovered and spatial cognition ability to be improved, but in the case of transplanting bone marrow mescenchymal stem cells transduced with the CCL5 siRNA, bone narrow mescenchymal stem cells does not improve memory function and spatial cognition ability.

The present invention provides a pharmaceutical composition for preventing or treating neurodegenerative diseases, including any one or two or more selected from the group consisting of a CCL5, a CCL5 expression regulator, and a CCL5 activator as an active ingredient.

Regarding to the pharmaceutical composition of the present invention, any one or two more selected from the group consisting of a CCL5, a CCL5 expression regulator, and a CCL5 activator may be included in 0.1 parts to 50.0 parts by weight, relative to 100 parts by weight of the total pharmaceutical composition. However, the range of the present invention is not limited thereto.

The pharmaceutical composition of the present invention may be prepared in any dosage form that is generally prepared in the relative art (for example: Literature [Remington's Pharmaceutical Science, latest version; Mack Publishing Company, Easton Pa.]). For example, a pharmaceutical composition for an oral administration, such as granules, an infinitesimal grain, powders, hard capsuia, soft capsula, syrups, an emulsion, suspension, and liquid formulation may be administrated. In addition, it may be possible to administer as injections for an intravenous administration, an intramuscular administration, and a subcutaneous administration, and a medicine composition for a parenteral administration, such as mucus formulation, suppository, a percutaneous absorbent, a transmucosal absorbent, collunarium, eardrops, instillations, inhalation, cream formulation, ointment, and catapiasma formulation.

The pharmaceutical composition according to the present invention may further include carrier, excipient, and diluents, which are suitable for economically using in a production process of the pharmaceutical composition.

carrier, excipient, and diluents that may be included in the pharmaceutical composition according to the present invention may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, minerals, and the like.

In the pharmaceutical composition according to the present invention, the amount of a CCL5 used may be depended on age, sex, and body weight of patient, but may be 0.01 mg/kg to 100 mg/kg, preferably 0.1 mg/kg to 10 mg/kg in one time or several times a day. In addition, such a dose may be increased or decreased depending on an administration route, a degree of the disease, sex, body weight, age, and the like. Accordingly, the range of the present invention is not limited to such a dose under any circumstances.

The present invention provides a food composition for preventing or treating neurodegenerative diseases, including any one or two or more selected from the group consisting of a CCL5, a CCL5 expression regulator, and a CCL5 activator as an active ingredient.

In addition, any one or two or more selected from the group consisting of a CCL5, a CCL5 expression regulator, and a CCL5 activator may be available as a main material, an additional material, or food additives of other foods.

The food composition according to the present invention may be diversely used in a functional food or drink, and the like. Examples of food that can be added with any one or two or more selected from the group consisting of a CCL5, a CCL5 expression regulator, and a CCL5 activator may include all kinds of foods, drinks, chewing gums, teas, vitamin complex, health functional foods, and the like.

In the food composition according to the present invention, any one or two or more selected from the group consisting of a CCL5, a CCL5 expression regulator, and a CCL5 activator may be generally added in 0.001% to 15% by weight in the food composition, and in the range of 0.002 g to 10 g and preferably 0.03 g to 1 g based on 100 ml in a health drink. However, the range of the present invention is not limited to the contents.

The health drink according to the present invention may include any one or two or more selected from the group consisting of a CCL5, a CCL5 expression regulator, and a CCL5 activator as an essential ingredient in the ratio suggested, but no specifically limit on a liquid ingredient, various flavoring agent, natural carbohydrates, and the like may be included as an additive ingredient, like a general drink.

Example of the natural carbohydrate may include a general sugar, such as monosaccharide, for example, glucose, fructose, and the like; disaccharide, for example, maltose, sucrose, and the like; and polysaccharide, for example, dextrin, cyclodextrin, and the like; and sugar alcohol, such as xylitol, sorbitol, and erythritol. Above this, a natural flavoring agent (thaumatin, stevia extract, for example, rebaudioside A, glycyrrhizin, and the like) and a synthetic flavoring agent (saccharine, aspartame, and the like) may be advantageously used as a flavoring agent.

Above this, the composition of the present invention may include various nutritional supplements, vitamins, mineral (electrolyte), a flavoring agent, such as a synthetic flavoring agent and a natural flavoring agent, a coloring agent, enhancers (cheese, chocolate, and the like), pectic acid and a salt thereof, alginic acid and a salt thereof, an organic acid, a protective colloid thickener, pH adjuster, a stabilizer, preservative, glycerin, alcohol, a carbonating agent that is used in carbonated drinks, and the like.

Hereinafter, exemplary embodiments of the present invention will now be described in detail with reference to the following Examples. However, the following Examples and Experimental Examples are only for illustrating the present invention, but the range of the present invention will not be limited to the following Examples and Experimental Examples.

### EXAMPLE

### 1. Experimental Method

### 1) Mouse Preparation

Transgenic mouse lines over-expressing the hAPP695swe (APPswe) and presenilin-1M146V (PS1) mutations, respectively, were generated at GlaxoSmithKline (Harlow, UK) by standard techniques on a C57BL/6 background (Charles River, UK). APPswe mice were backcrossed onto a pure C57BL/6 background before crossing with PS1 mice to produce double heterozygous mutant mice (APP/PS1). Green fluorescent protein (GFP) transgenic (C57BL/6-Tg (ACTB-EGFP) 1Osb/J) mice were purchased from the Jackson Laboratory (Bar Harbor, Me., USA).

### 2) AFP/PS1-GFP Chimeric Mice

6-month-old APP/PS1 mice (recipients) were exposed to 10 Gy whole body irradiation (2×5 Gy) except in the brain, and to a 5 Gy head irradiation. (1×5 Gy) . Donor BM cells (1×10⁷ per mouse) derived from GFP mice were administrated via tail vein to each recipient. Transplanted mice were given drinking water complemented with 0.2 mg/ml trimethoprim and 1 mg/ml sulfamethoxazole for 2 weeks. Five weeks after the BM transplantation, chimeric mice were confirmed by blood smears from tail clippings for the presence of GFP.

### 3) Cell Isolation and Culture

Tibias and femurs were dissected from 4- to 6-week-old C57BL/6mice. BM was harvested, and single-cell suspensions were obtained using a 40 μm cell strainer (Becton-Dickinson Labware, Franklin Lakes, N.J.). Approximately 107 cells were plated in 75-cm² flasks containing MesenCult™ MSG Basal Medium and Mesenchymal Stem Cell Stimulatory Supplements (Stem Cell Technologies, Inc) with antibiotics. The cell cultures were grown for 2 weeks, and the plastic-adherent population (BM-MSCs) was used for subsequent experiments. BV2 cells were cultured in DMEM (Gibco) supplemented with 10% fetal bovine serum (FBS, Gibco). Primary microglia was prepared from 3-day-old C57BL/6 mice pups.

### 4) Knockdown of GCLS using siRNA

Small interference RNA (siRNA) oligonucleotides for CCL5 and scrambled sequence siRNA (siCONTROL) serving as a control were obtained from Dharmacon (Chicago, Ill.). BM-MSCs were seeded in a 10-cm tissue culture dish. On the following day, transfection was performed using lipofectamine 2000 reagent (Invitrogen, Carlsbad, Calif.). Two days post-transfaction, efficiency of knockdown by siRNA was assessed by real-time RT-PCR.

### 5) Transwell Chamber Migration Assay

The transwell chamber migration assay was carried out using transwell cell culture inserts (Corning, 5-μM pore size) or the Cytoselect™ 24-well cell migration assay kit (CELL BIOLABS, INC).

### 6) Cytokine Arrays

For cytokine analysis, we used a mouse cytokine antibody array kit (R & D systems) following the manufacturer's instructions. The array membranes were incubated with blocking buffer followed by mixtures of BM-MSC CM with or without 10 μM Aβ42 stimulation, and then a detection antibody cocktail at 4° C. overnight.

### 7) Treatment Protocol

Three days before the first injection with BM-MSCs, the mice were anesthetized with a combination of 100 mg/kg ketamine and 10 mg/kg xylazine, and a stainless steel cannula was implanted in the animal's hippocampus using a stereotaxic frame (David Kopf Instrument, Tujunga, Calif.). BM-MSC suspensions, CCL5 knockdown BM-MSCs, or PBS were transplanted biweekly for 1 month (n=10 per group). APP/PS1-GFP chimeric, mice (n=10, per group) were treated by the same protocol.

### 8) Tissue Preparation

Mice were anesthetized with 2.5% avertin in PBS. Animals were immediately cardiac perfused with 4% paraformaldehyde in PBS. After perfusion, brains were removed, postfixed overnight at 4° C., incubated in 30% sucrose at 4° C. until equilibrated, and embedded in OCT compound for frozen section. Sequential 30 μm coronal sections were taken on a cryostat (CM3050S; Leica) and stored at −20° C.

### 9) Immunohistochemistry

Free-floating sections were incubated for 1 hr in PBS containing 5% normal goat serum, 2% BSA, and 0.4% Triton X-100. In the same buffer solution, the sections were then incubated for 24 hr in primary antibodies at 4° C. The following antibodies were used: 20G10 (mouse, diluted 1:1000), G30 (rabbit, diluted 1:1000), anti Iba-1 (rabbit, diluted 1:500, Wako), anti 6E10 (mouse, diluted 1:500, Signet), anti NEP (goat, diluted 1:10, R&D system) and anti IL-4 (goat, diluted 1:250, SantaCruz). For visualization, the primary antibody was developed by incubating with Alexa Fluor 488-, 594- or 633-conjugated secondary antibodies. For some experiments, tissue sections from APP/PS1-GFP chimeric mice were stained with combinations of the following primary antibodies: goat anti-IL-4 (1:250) and rabbit anti-Iba-1 (1:500), followed by the corresponding Alexa 546 and Alexa 633-conjugated secondary antibodies.

### 10) Quantitative Real-Time FCR

RNA was extracted from the brain homogenates and cell lysates using RNeasy Lipid Tissue Mini kit or RNeasy Plus Mini Kit (Qiagen, Korea, Ltd) according to the manufacturer's instructions. cDNA was synthesized from 5 μg of total RNA using a commercially available kit from Clontech (Mountain View, Calif.). Quantitative real-time PGR was performed using a Corbett research RG-6000 real-time PGR instrument, and a one-step program: 95° C., 10 min; 95° C., 10 sec, 58° C., 15 sec, 72° C., 20 sec for 40 cycles. The following primers were used: CXCL1 (forward: 5′-CACAAAATGTCCAAGGGAAG-3′ (SEQ ID NO.: 1), reverse: 5′-GCGAAAAGAAGTGCAGAGAG-3′ (SEQ ID NO.: 2)), Macrophage colony-stimulating factor (M-CSF) (forward: 5′-TTCCACCTGTCTGTCCTCAT-3′ (SEQ ID NO.: 3), reverse: 5′-AGTCTGTCTTCCACCTGCTG-3′ (SEQ ID NO.: 4)), macrophage inflammatory protein-1β□ (MIP-1β□□ (forward: 5′-ACGGGGGTCAATTCTAAG-3′ (SEQ ID NO.: 5), reverse: 5′-GCCATTCCTGACTCCACA-3′ (SEQ ID NO.: 6)), MIP-2 (forward: 5′-ACATCTGGGCAATGGAATTA-3′ (SEQ ID NO.: 7), reverse: 5′-TGAACAAAGGCAAGGCTAAC-3′ (SEQ ID NO.: 8)), CCL5 (forward: 5′-AAGCAATGACAGGGAAGCTA-3′ (SEQ ID NO.: 9), reverse: 5′-CAATCTTGCAGTCGTGTTTG-3′ (SEQ ID NO.: 10)), Insulin degrading enzyme (IDE) (forward: 5′-GAAGACAAACGGGAATACCGTG-3′ (SEQ ID NO.: 11), reverse: 5′-CCGCTGAGGACTTGTCTGTG-3′ (SEQ ID NO.: 12)), Neprilysin (NEP) (forward: 5′-GAAATTCAGCCAAAGCAAGC-3′ (SEQ ID NO.: 13), 5′-GATTTCGGCCTGAGGAATAA-3′ (SEQ ID NO.: 14)), Matrix metalloproteinase 9 (MMP9) (forward: 5′-GCCATGCACTGGGCTTAGAT-3′ (SEQ ID NO.: 15), reverse: 5′ -TCTTTAITCAGAGGGAAGCCCTC-3′ (SEQ ID NO.: 16)), TNF-α□ (forward: 5′-GCTCCAGTGAATTCGGAAAG-3′ (SEQ ID NO.: 17), reverse: 5′-GATTATGGCTCAGGGTCCAA-3′ (SEQ ID NO.: 18)), IL-1β□ (forward: 5′-CCCAAGCAATACCCAAAGAA-3′ (SEQ ID NO.: 19), reverse: 5′-GCTTGTGCTCTGCTTGTGAG-3′ (SEQ ID NO.: 20)), IL-4 (forward: 5′-ATCCATTTGCATGATGCTCT-3′ (SEQ ID NO.: 21), reverse: 5′-GAGCTGCAGAGACTCTTTCG-3′ (SEQ ID NO.: 22)), YM-1 (forward: 5′-AGAGCAAGAAACAAGCATGG-3′ (SEQ ID NO.: 23), reverse: 5′-CTGTACCAGCTGGGAAGAAA-3′ (SEQ ID NO.: 24)), and GAPDH (forward: 5′-TTGCTGTTGAAGTCGCAGGAG-3′ (SEQ ID NO.: 25), reverse: TGTGTCCGTCGTGGATCTGA-3′ (SEQ ID NO.: 26)).

### 11) Western Blot Analysis

Brain hippocampi were isolated from mice. The brain tissues were weighed and sonicated in 10× volume of RIPA buffer (20 mM Iris, pH 7.4, 150 mM NaCl, 1% NP-40, 2 mM EDTA, 0.1% Na deoxycholate, 0.1% SDS, 50 mM NaF, 1 mM PMSF, 1 mM Na₃VO₄, 10 mg/ml aprotinin and 10 mg/ml leupeptin) plus protease inhibitors. Protein concentrations were determined using Bradford technique (Bio-Rad, Hercules, Calif.). Equal amounts of proteins (80 μg) were fractionated by SDS-PAGE, transferred to PVDF membranes. Membrane was incubated with anti-NEP (1:1000, R&D Systems) Membranes were developed by enhanced chemiluminescence detection system (ECL; Amersham Biosciences).

### 12) Flow Cytometric Analysis

APP-PS1 GFP chimeric mice were perfused with 30 ml of PBS. Hippocampal and cortical tissues were carefully dissected and dissociated using RPMI 1640 (Gibco, no phenol red) containing 2 mM L-glutamine, dispase and collagenase type 3 (Sigma-Aidrich). The enzymes were inactivated by addition of 20 ml of Ca²⁺/Mg²⁺-free Hank's balance salt solution (BBSS) containing 2 mM EDTA and 2% FBS, followed by trituration using pipettes of decreasing diameter. Cells were pelleted and resuspended in RPMI 1640/L-glutamine and mixed with physiologic Percoll (Sigma-Aldrich) and centrifuged at 85×g for 45 min. The cells were then incubated with anti-mouse Cd11b-coated microbeads (Miltenyi Biotec) for 20 min at 12° C. The cell-bead mix was then washed to remove unbound beads. The bead-cell pellet was resuspended in PBS/0.5% BSA/2 mM EDTA and passed over a magnetic MACS Cell Separation column (Miltenyi Biotec) following the manufacturer's instructions. CD11b-positive cells were eluted by removing the column from the magnetic holder and pushing PBS/BSA/EDTA through the column with a plunger. The cells were washed and stained PE-conjugated CD45 (Beeton Dickinson). Isotype-matched antibodies served as controls.

### 13) ELISA

Quantification of CCL5, TNF-α, and IL-4 protein levels were determined using commercially available mouse ELISA kits (CCL5, R&D systems, TNF-α, USCN life and IL-4, Raybiotec). Standard curves were prepared using purified cytokine standards.

### 14) Immunofluorescence Staining of F-Actin

BV2 cells were placed on glass slides and allowed to adhere overnight. CM from BM-MSCs with or without Aβ□ stimulation was added to the cells the following day, and incubation was continued, for an additional 24 hr. Cells were fixed and processed for immunofluorescence staining of F-actin. (i.e., 0.1% Triton for 5 min, washed twice with PBS, and blocked with blocking buffer for 15 min). Phalloidin-tetramethylrhodamine B isothiocyanate (Sigma-Aldrich) was used at a final concentration of 50 ng/ml. DAPI was used for nuclear staining.

### 15) Behavioral Test

We used the Morris water maze (MWM) task to assess spatial memory performance. A submerged Piexiglas platform (10 cm diameter; 6-8 mm below the surface of the water) was located at a fixed position throughout the training session. A single probe trial, in which the platform was removed, was performed after the hidden platform task had been completed (day 11). Each mouse was placed into one quadrant of the pool and allowed to swim for 60 sec.

### 2. Experimental Results

### 1) CM from BM-MSCs Stimulated by Aβ□induces Migration of Monocyte and Microglia In Vitro

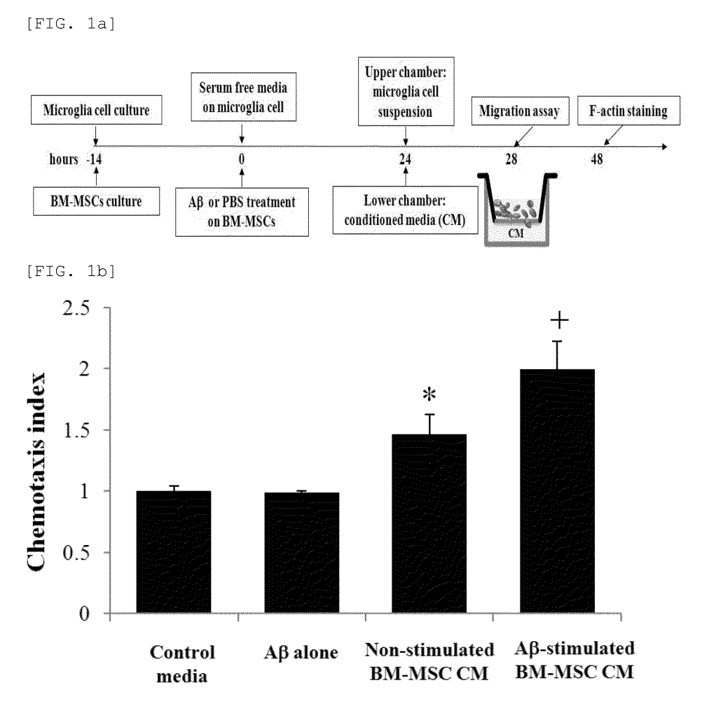

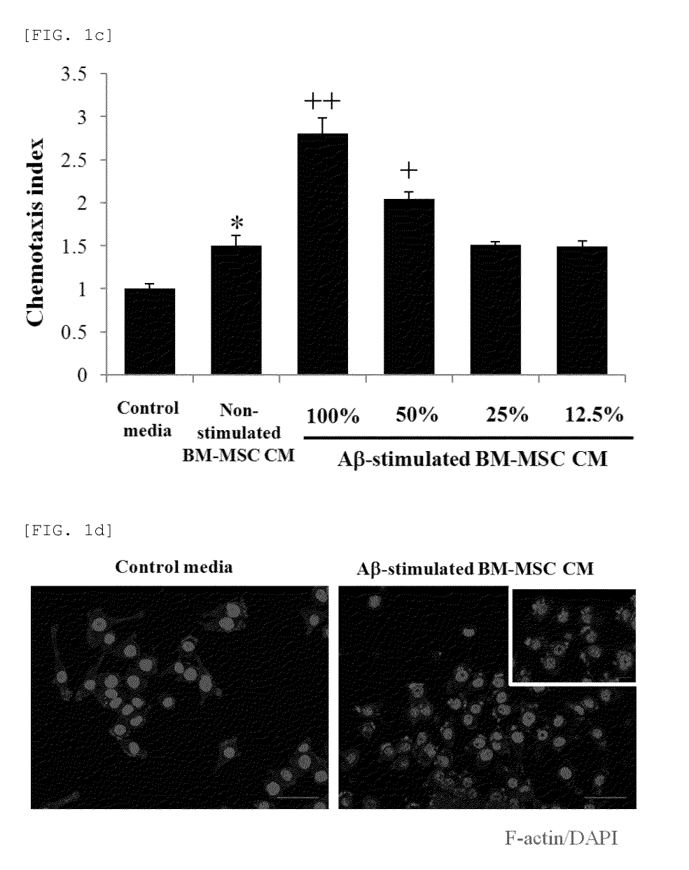

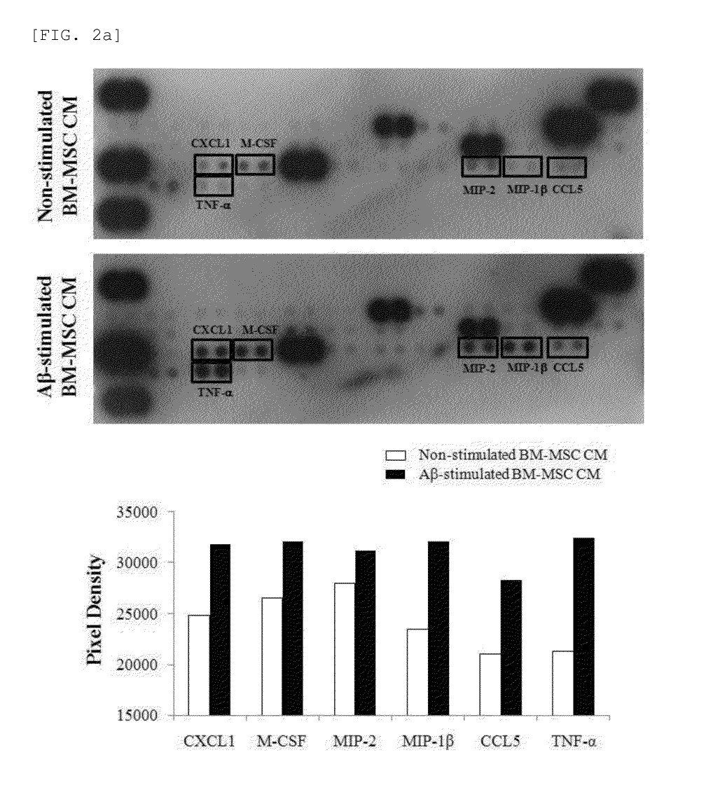

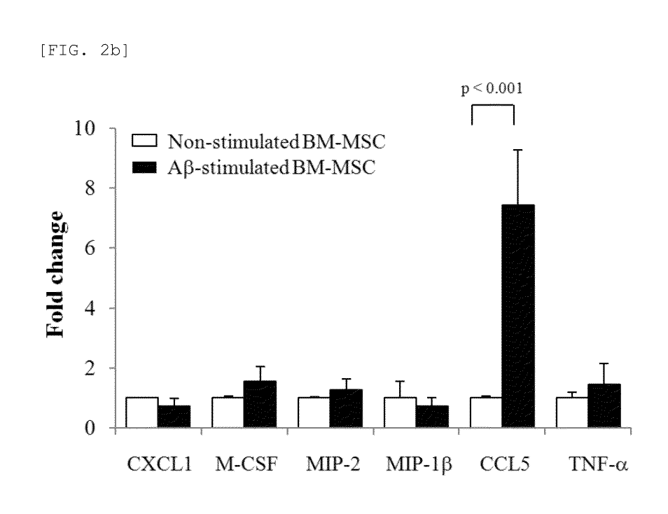

To examine whether soluble factors released from BM-MSCs exhibited chemoattractive effects following exposure to Aβ, a transwell migration assay was performed using BM-MSC CM (FIG. 1). Briefly, CM was prepared by treatment of BM-MSCs with 10 μM Aβ for 24 h, and then added to the bottom chamber of transwell culture dishes where the upper chamber contained a monocyte/microglia (BV2) cell suspension. The number of BV2 cells that moved (migrated) to the lower chamber was then quantified. We first found that BV2 microglia migrated to wells containing BM-MSC CM non-stimulated with Aβ, as well as to non-β stimulated CM from NIH 3T3 control cells. Notably, the BM-MSC CM significantly enhanced microglia migration compared to control media (from non-cultured BM-MSC) and control cell CM (p<0.005, vs control media).

To examine this effect further, we stimulated BM-MSCs with 10 μM aggregated Aβ□42 for 24 hr, and then used the CM from these cells for the migration assays (FIG. 1). First, non Aβ-stimulated BM-MSC CM induced migration of both monocyte and BV2 cells compared with control media (from non-cultured BM-MSC) and 10 μM Aβ□42 alone treatment in control media (p<0.05, vs control media and Aβ□42 alone, FIG. 1b). Also, Aβ-stimulated BM-MSC CM induced migration of both monocyte and BV2 microglial cells compared with non-stimulated BM-MSC CM (p<0.05, vs non-Aβ□ stimulated. BM-MSC CM, FIG. 1b). Dose-dependent, migration of monocyte and BV2 microglia in response to Aβ-treared BM-MSC CM was observed (FIG. 1c).

We examined the effect of Aβ□treated BM-MSC CM on cytoskeletal reorganization in BV2 microglia. Actin stress fiber formation was significantly increased in BV2 cells when they were stimulated with Aβ□treated BM-MSC CM compared to control (from non-cultured BM-MSC) media (FIG. 1d). Although BV2 microglia exposed to non Aβ-stimulated BM-MSC CM also exhibited actin stress fiber formation, the number of lamellipodia was less than in cells exposed to Aβ-stimulated BM-MSC CM (data not shown).

### 2) Cytokine Expression Profile of BM-MSC CM after Aβ□Exposure

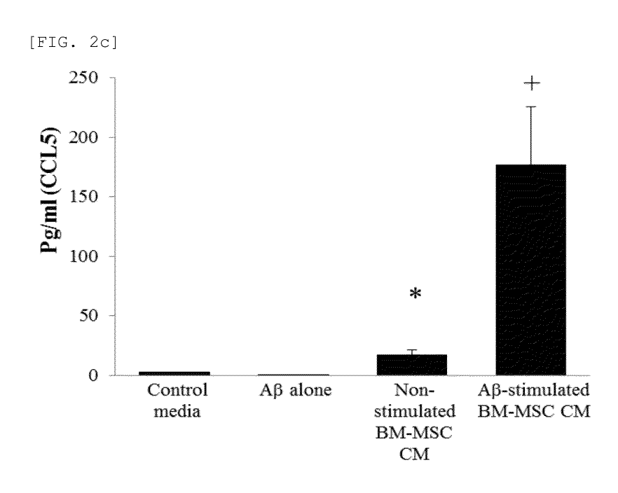

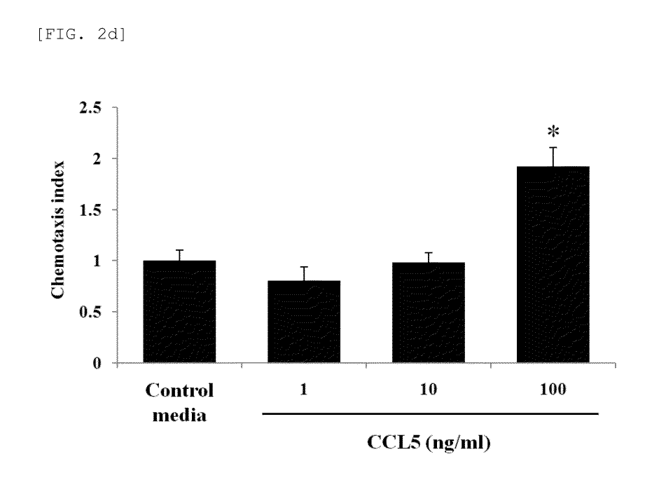

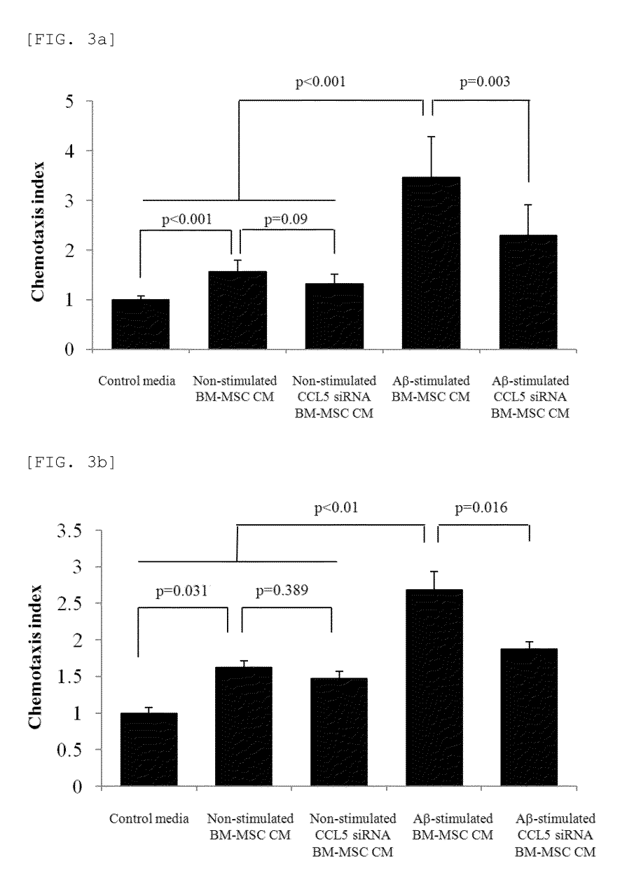

In order to identify the chemotactic cytokines that were upregulated in BM-MSCs after Aβ stimulation, we screened and compared the CM of non- and Aβ-stimulated BM-MSCs for 40 different secreted cytokines using an antibody-based mouse cytokine array. The cell-free supernatant of Aβ-stimulated BM-MSCs induced stronger signals in 6 array spots in comparison to the supernatant of non-stimulated BM-MSCs. CM derived from BM-MSCs exposed to Aβ□showed higher levels of CXCL1, M-CSF, MIP-2, MIP-1β, CCL5 and TNF-α (FIG. 2a).

The increased levels of cytokines were confirmed by quantitative real-time RT-PCR analysis of RNA prepared from the non- and Aβ-treated BM-MSCs (FIG. 2b). Of the selected cytokines, only CCL5 levels were significantly elevated in the mRNA of BM-MSCs after Aβ□stimulation.

To confirm the secretion of CCL5 in BM-MSCs exposed to Aβ, we performed ELISA using the non- and Aβ-stimulated BM-MSCs CM. The results showed that the CCL5 protein levels were higher in the Aβ-stimulated BM-MSC CM compared to non-stimulated BM-MSC CM (p<0.001, FIG. 2c).

We subsequently examined whether the CCL5 derived from the BM-MSCs could function as a chemoattractant and might be responsible for the monocyte and microglia migration we had observed. At 100 ng/ml, recombinant murine CCL5 significantly promoted monocyte and BV2 microglial migration when compared with control media (p<0.05) (FIG. 2d).

### 3) Soluble CCL5 Derived from BM-MSCs and Activated by Aβ is a Critical Factor that Promotes Monocyte and Microglia Migration

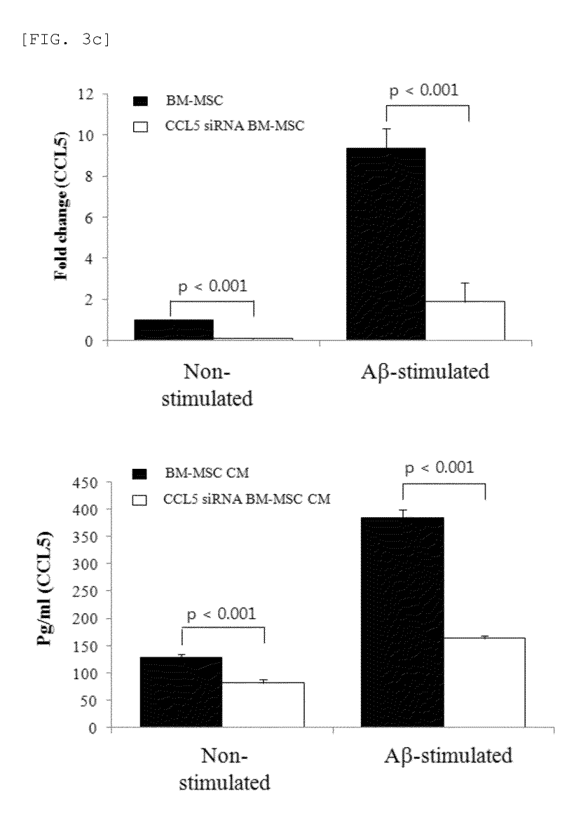

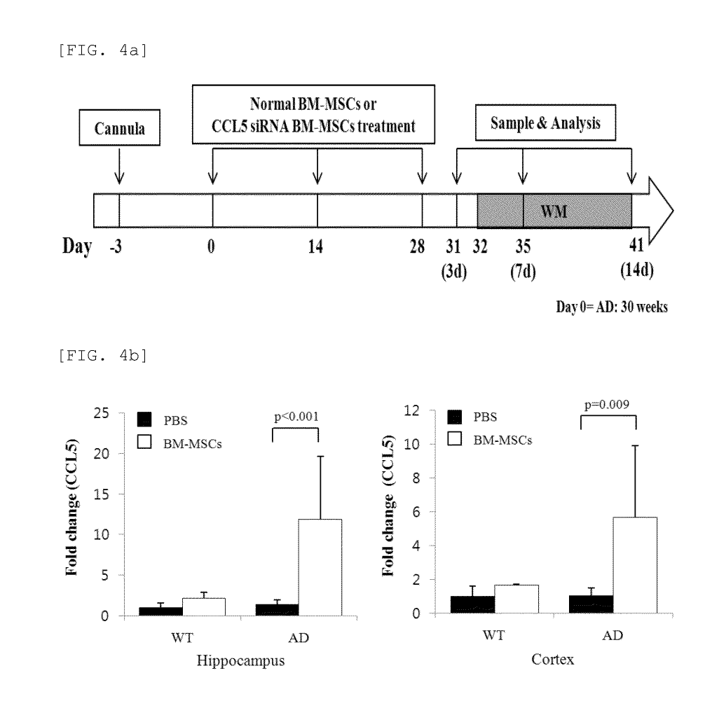

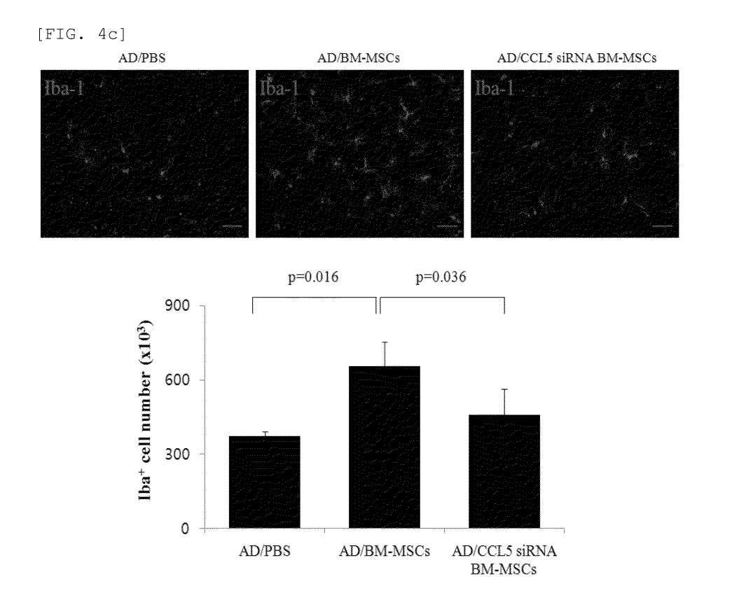

To further confirm that CCL5 derived from BM-MSCs was important in promoting monocyte/microglia migration, we used siRNA to knockdown CCL5 expression in BM-MSCs. 48 hr after transfection with a construct expressing CCL5 siRNA, the CCL5 mRNA and protein content in BM-MSCs were decreased 90% and 39%, respectively, compared to control siRNA treated BM-MSCs (p<0.001). We also observed decreased CCL5 mRNA (80% decrease) and protein levels (58% decrease) after Aβstimulation of CCL5 siRNA treated BM-MSCs compared to control siRNA treated BM-MSCs (p<0.001) (FIG. 3c).

CM was collected with or without. Aβ stimulation from BM-MSCs and CCL5 knockdown BM-MSCs, and monocyte and microglia migration assays were then performed. CM from non Aβ-stimulated BM-MSCs induced significant BV2 cells migration compared to the control (from non-cultured BM-MSC) media (p<0.001, FIG. 3a). This effect was lower when CM of CCL5 knockdown BM-MSCs was tested (p<0.05, vs BM-MSCs CM, FIG. 3a). Similar effects were observed when primary microglia was tested (FIG. 3b).

### 4) Soluble CCL5 Derived from BM-MSCs Following Transplantation is a Critical Factor to Recruit Endogenous Microglia in the AD Mouse Brain

In order to examine the in vivo effects of BM-MSCs on monocyte and microglia migration in AD, we used APP/PS1 double transgenic mice with Aβ□depositions in cortex and hippocampus.

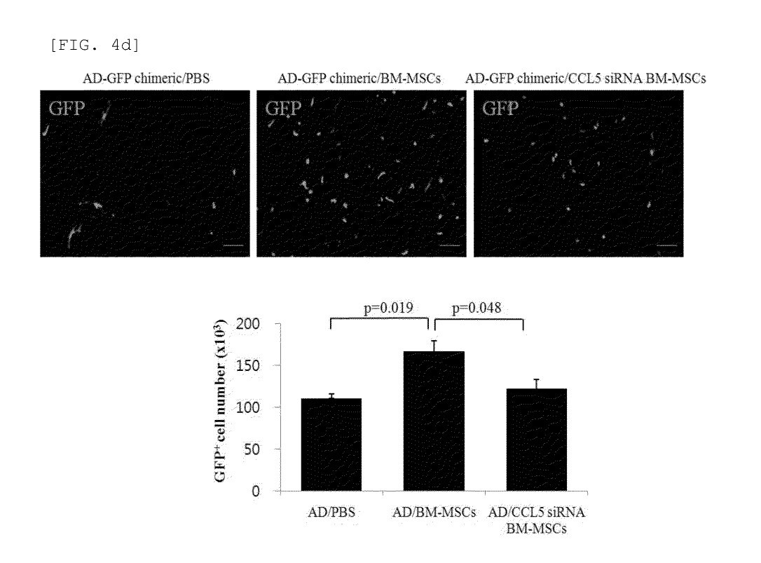

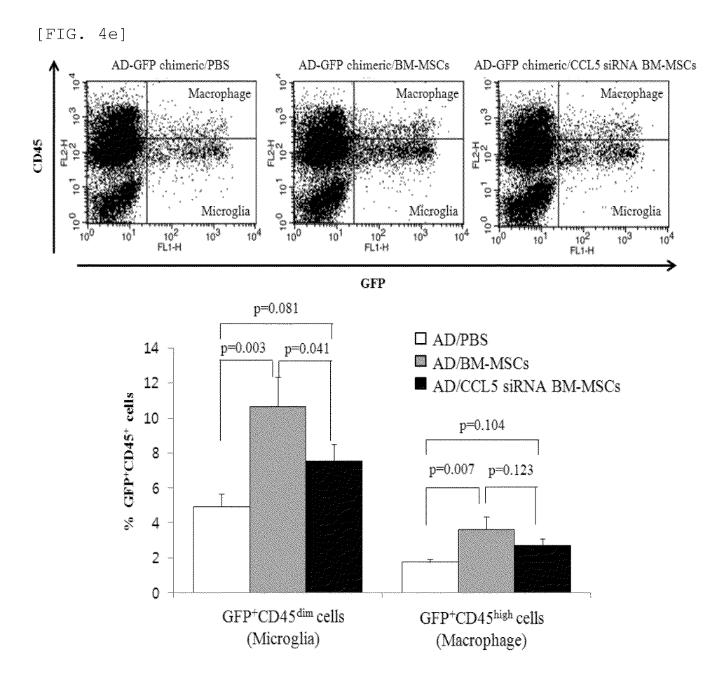

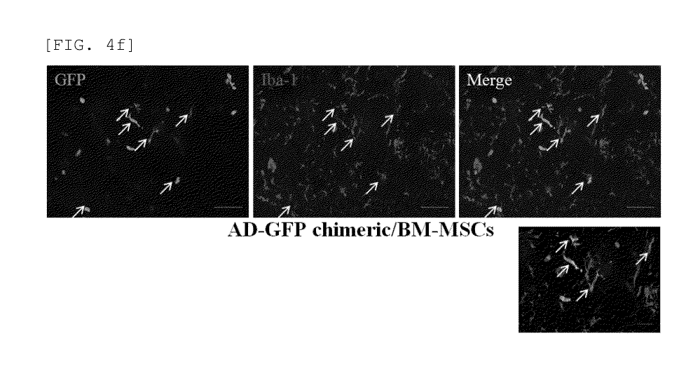

The treatment protocol is described in FIG. 4a. At 2 weeks after the last BM-MSC transplantation, we observed that CCL5 mRNA was significantly increased in BM-MSC transplanted APP/PS1 mice compared with PBS infused counterparts (p<0.001, FIG. 4b). The increased expression of CCL5 observed in the BM-MSC treated APP/PS1 mice was more significant in the hippocampus than the cortex.

To examine whether the increased CCL5 levels were associated with microglia activation, we first investigated recruitment of microglial cells in the hippocampus by counting Iba-1 positive ceils using stereological analysis in the treated and non-treated APP/PS1 mice. In BM-MSC treated mice, the number of microglia was significantly increased compared with PBS treated mice (p=0.016, vs AD/PBS, FIG. 4c). However, in mice transplanted with CCL5 knockdown BM-MSCs, microglia recruitment was significantly lower than in control BM-MSC infused mice (p=0.036, vs AD/BM-MSCs, FIG. 4c).

We constructed chimeric mice by irradiating 6-month-old APP/PS1 mice and intravenously injecting BM cells collected from GFP mice. At 5 weeks after the BM transplantation, chimeric mice were confirmed by the presence of GFP in BM cells and peripheral blood monocyte using flow cytometric analysis. At 2 weeks after the last BM-MSC injection, brain sections were taken and the number of GFP positive cells in the hippocampal region was estimated using stereological analysis. As a result, injection of BM-MSCs led to a significant increase of GFP positive cells (p=0.019, vs AD-GFP chimeric/PBS, FIG. 4d). However, infusion of CCL5 knockdown BM-MSCs reduced these effects of BM-MSCs (p=0.048, FIG. 4e).

We separated CD11b positive cells by MACS and by flow cytometry analysis using brain suspensions from AD-GFP chimeric mice 14 days after the last BM-MSC transplantation. The relative levels of CD45 can distinguish microglia (CD45 intermediate) from macrophage (CD45 high). In our result, the percentage of GFP-positive CD45-intermediate (GFP⁺/CD45^{dim}) microglia obtained from separated CD11b positive cells were increased in the BM-MSC treated GFP chimeric mice compared with the PBS treated group.

We first determined the contents of CCL5 in the hippocampus at earlier time points 3 and 7 days after the last BM-MSC or CCL5 knockdown BM-MSC treatment using quantitative real time RT-PCR and ELISA assay. CCL5 contents derived from BM-MSC after transplantation showed time dependent decreasing trends.

To further study the role of CCL5 in the AD mouse brain, we therefore examined earlier time points after BM-MSC treatment. At 3 days after BM-MSC infusion in GFP chimeric APP/PS1 mice, the GFP+/CD45^{dim} microglia were significantly increased in the BM-MSC treated mice compared with the PBS treated group (p=0.003, vs AD-GFP chimeric/PBS, FIG. 4e). GFP-positive and CD45-high macrophage (GFP⁺/CD45^{high}) were also increased in the BM-MSC transplanted mice than the PBS infused mice (p=0.007, vs AD-GFP chimeric/PBS, FIG. 4e). However, mice treated by BM-MSCs with CCL5 siRNA knockdown showed significantly reduced GFP+/CD45^{dim} microglia and slightly decreased GFP⁺/CD45^{high} macrophages.

We also analyzed AD-GFP chimeric/BM-MSCs mice 14 days after the last transplantation by histology (FIG. 4f). Microscopic investigation of the brain revealed that numerous BM-derived cells (GFP positive cells) expressed Iba-1, a marker for microglia, confirming the differentiation of BM-derived cells into microglia.

### 5) Soluble CCL5 Derived from BM-MSCs Modulates the Microglial Activation Status in APP/FS1 Mice

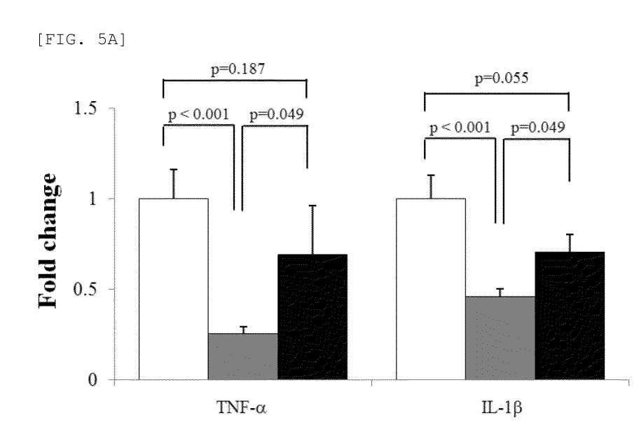

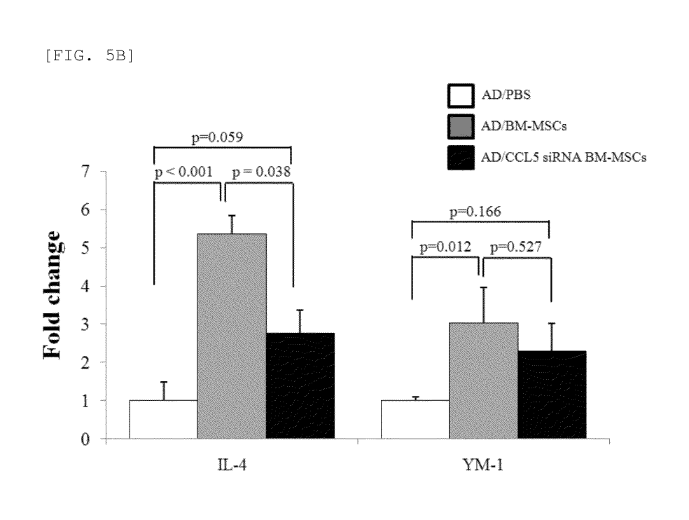

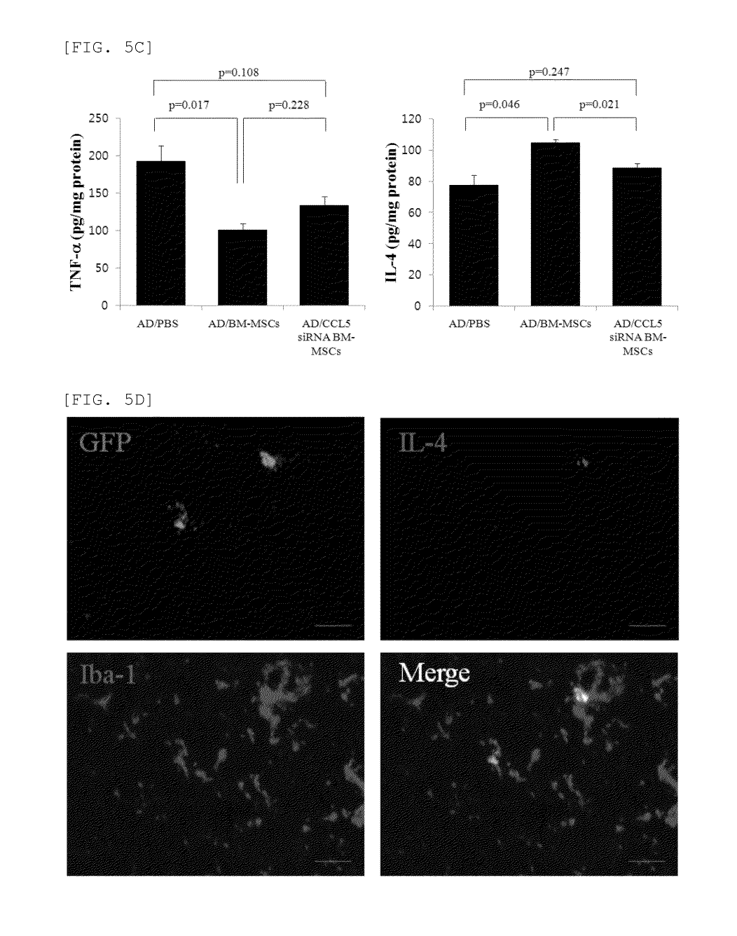

Our APP/PS1 mice showed increased levels of pro-inflammatory cytokines at 9 months of age compared with normal mice. At 3 days after BM-MSC treatment, we found BM-MSC transplanted mice exhibited a 4 fold decrease in TNF-α and a 2 fold decrease in IL-1 compared to PBS-treated APP/PS1 mice (p<0.001, vs AD/PBS, FIG. 5a). However, treatment with CCL5 knockdown BM-MSCs did not show decreased TNF-α□ and IL-1β□expression in the hippocampus (p=0.049, vs AD/BM-MSCs, FIG. 5a). While the hippocampal brains of BM-MSC transplanted APP/PS1 mice had significantly increased levels of the alternative microglia markers, IL-4 (p<0.001, vs AD/PBS) and YM-1 (p=0.012, vs AD/PBS), blockade of CCL5 expression in BM-MSCs significantly inhibited the induction of IL-4 expression (p=0.038, vs AD/BM-MSCs, FIG. 5b). APP/PS1 mice treated by transplantation of BM-MSCs with siRNA CCL5 knockdown slightly inhibited the induction of YM-1 expression compared with the BM-MSC treated group, although this did not reach statistical significance (p=0.527, vs AD/BM-MSCs, FIG. 5b).

To confirm these effects, we measured the TNF-α and IL-4 protein content in the hippocampus by ELISA. As shown in FIG. 5C, TNF-α□ was lower (p=0.017, vs AD/PBS) and IL-4 higher (p=0,046, vs AD/PBS) in the BM-MSC treated mice compared to the PBS-treated APP/PS1 mice. When CCL5 was knocked down in the BM-MSCs by siRNA, these outcomes were changed (FIG. 5c). Immunofluorescent images in AD-GFP chimeric mice showed that the BM-derived microglia expressed IL-4 at 14 days after the last BM-MSC treatment (FIG. 5d).

### 6) Microglia Recruited by BM-MSC-Derived CCL5 can Reduce Aβ Deposition by Expression of Aβ-Degrading Enzymes in AFF/PS1 Mice Brain

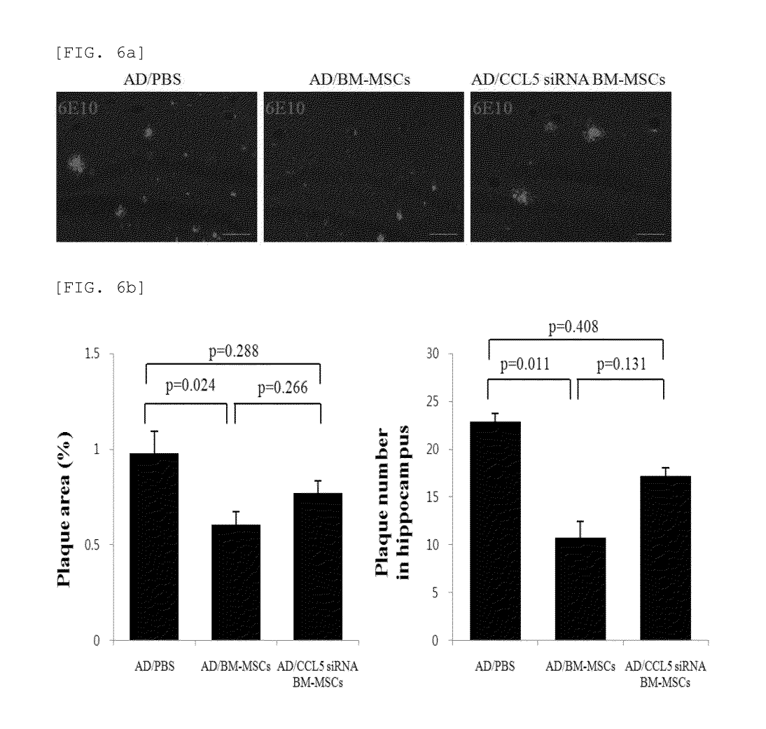

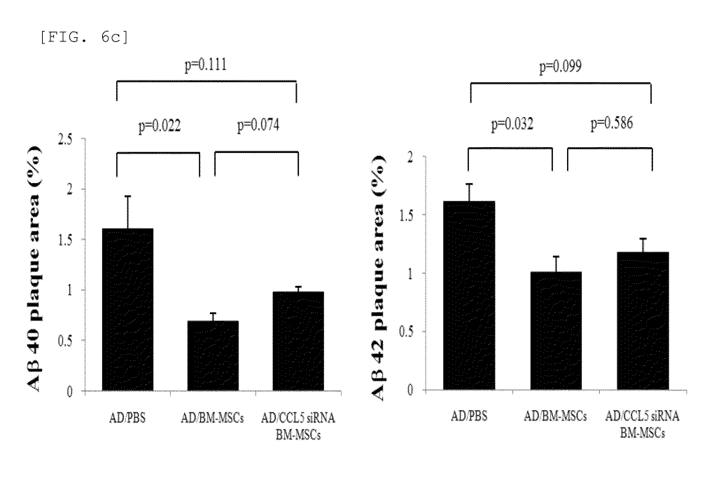

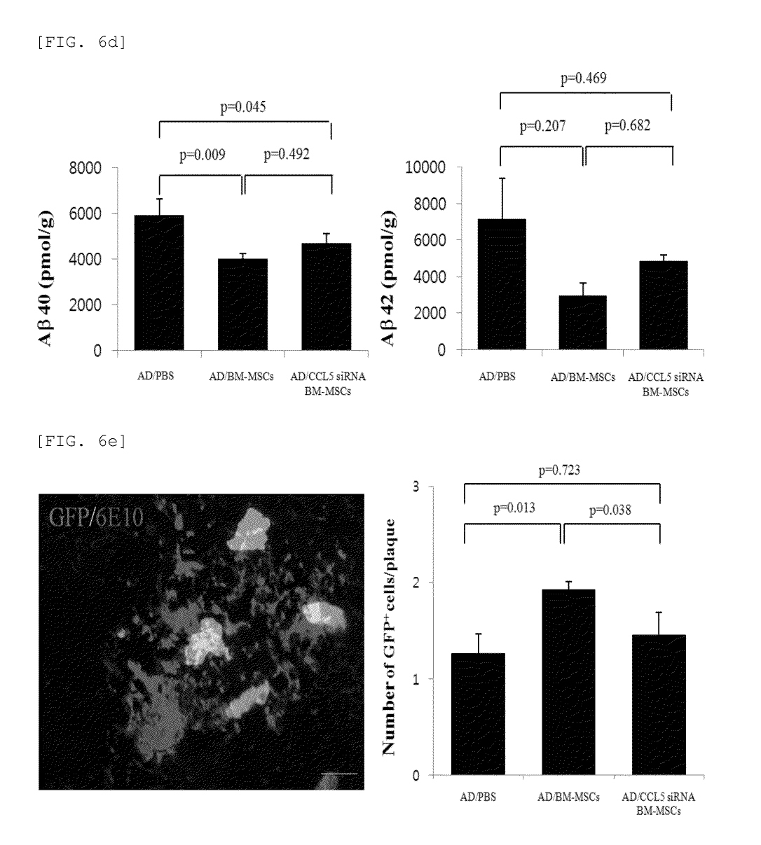

To analyze the effects of BM-MSC-derived CCL5 on Aβ□load in the brain, we first determined the Aβ□profile using 6E10 immunostaining analysis in treated APP/PS1 mice. We found the deposition of Aβ□ to be markedly reduced following BM-MSC transplantation (p<0.05, vs AD/PBS, FIGS. 6a and 6b) in the hippocampus of APP/PS1 mice.

Although, the Aβ□deposits also were slightly decrease in CCL5 knockdown BM-MSC treated mice compared with the PBS treatment group, the difference did not reach significance (p>0.05, vs AD/PBS, FIGS. 6a and 6b).

We further confirmed the role of CCL5 derived from BM-MSCs in Aβ deposition by using Aβ□40 and 42 immunohistochemical analyses and ELISA assays in the APP/PS1 mice. The contents of both Aβ□40 and 42 were significantly reduced following BM-MSCs treatment (p<0.05, vs AD/PBS, FIGS. 6c and 6d), and these effects were partially negated by knockdown of CCL5 gene prior to infusion of the cells (FIGS. 6c and 6d).

To determine the relationship between BM-derived cells and Aβ deposits after transplantation with BM-MSCs or BM-MSCs expressing CCL5 siRNA, BM-derived cells and Aβ deposits were immunostained and analyzed in AD-GFP chimeric mice. We clearly observed the co-localization of Aβ (6E10) and BM-derived cells (GFP positive). Recruitment of BM-derived cells to Aβ deposits (number of GFP positive cells/Aβ□plaque) appeared to be enhanced in BM-MSC treated mice compared with PBS treated mice (p=0.013, vs AD/PBS; FIG. 6e), but were significantly reduced in chimeric mice treated with BM-MSCs transduced with CCLo siRNA (p=0.038, vs AD/BM-MSCs, FIG. 6e).

We next analyzed the expression of Aβ degrading enzymes that are known to be released by microglia. Notably, we observed significantly increased levels of NEP and MMP9 in the hippocampal regions of AD mice treated with BM-MSCs compared to PBS infused mice. However, the expression of these enzymes was significantly decreased in the AD mice treated with BM-MSCs transduced with CCL5 siRNA (FIG. 6f). The levels of IDE also showed similar change patterns as NEP and MMP9, but this did not reach statistical significance.

To confirm these effects, we examined the levels of NEP in the hippocampus of AD mice by western blot analysis. As shown in FIG. 6g, NEP was significantly increased in the BM-MSC treated mice compared to the PBS-treated APP/PS1 mice. When CCL5 was knocked down in the BM-MSCs by siRNA, these outcomes were decreased (FIG. 6g).

To know whether the increased expression of NEP after BM-MSC treatment was associated with the migration of BM-derived cells into the brain, we performed NEP immunostaining using brain sections of AD-chimeric mice. We found that the GFP positive BM-derived cells expressed NEP at 14 days after the last BM-MSC transplantation (FIG. 6h).

### 7) Released CCL5 Following BM-MSC Transplantation into Aβ-Deposited Brain Improves Behavioral Abnormalities of APP/PS1 Mice

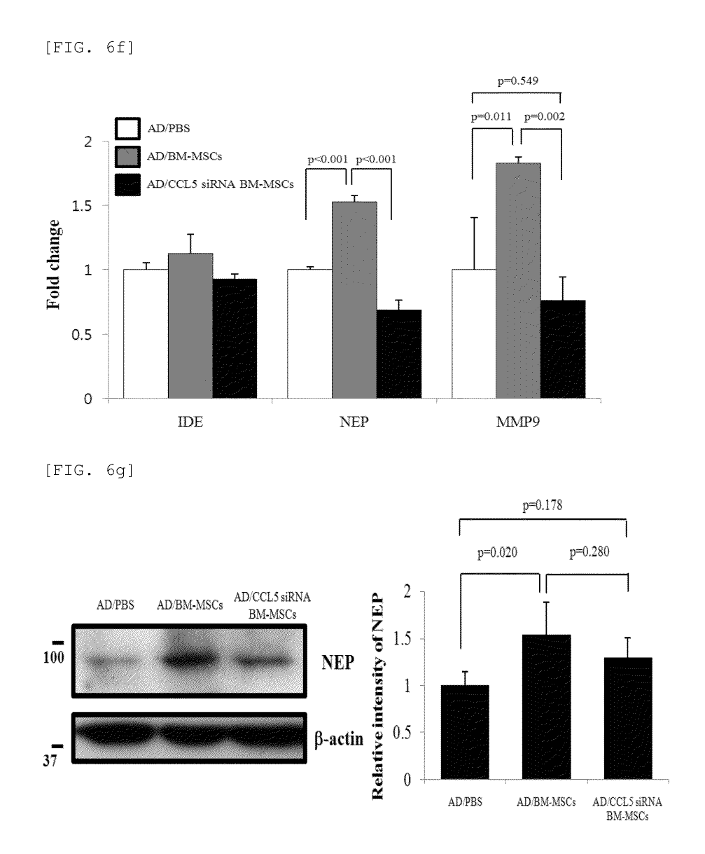

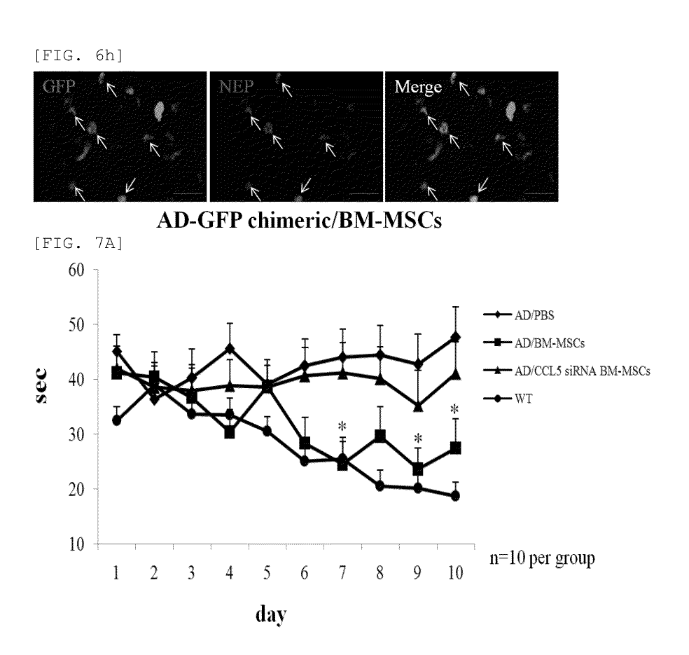

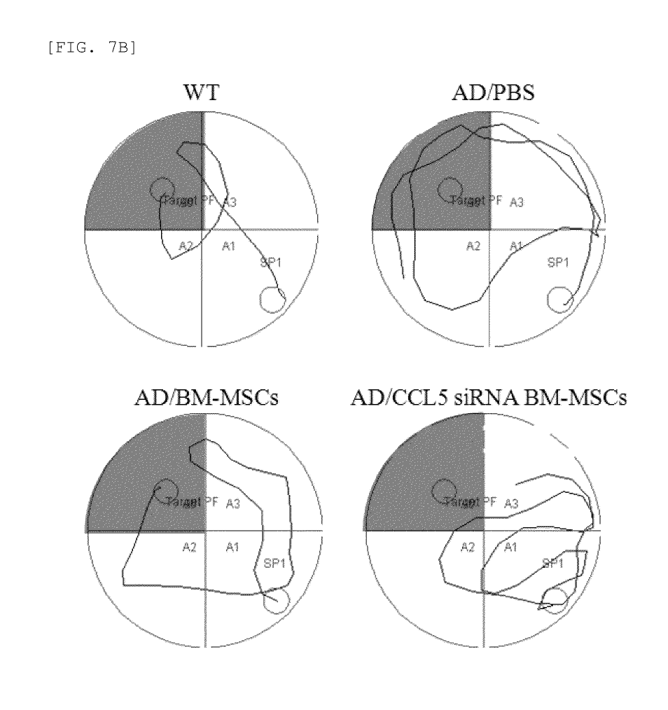

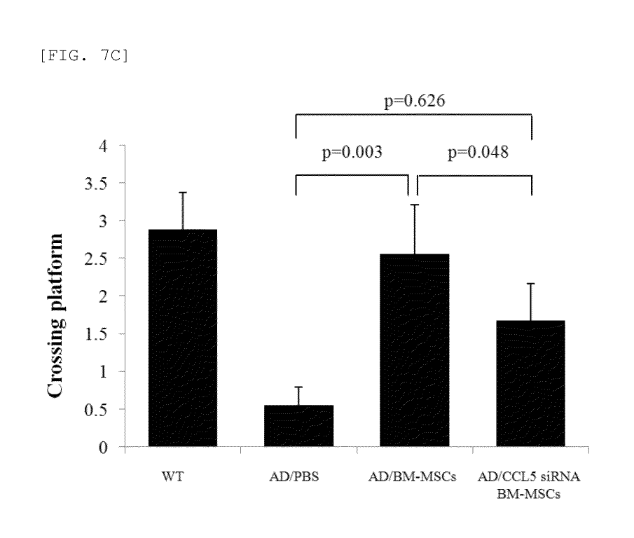

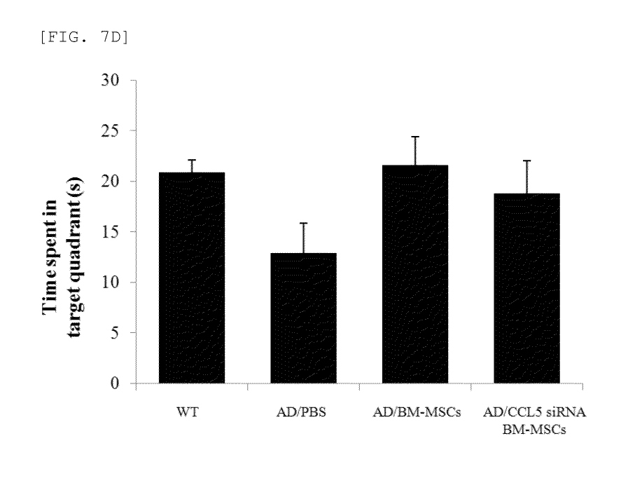

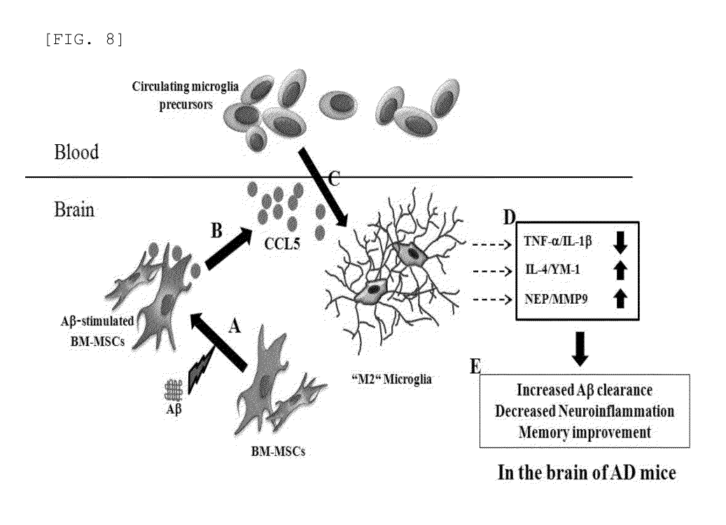

To address the role of BM-MSC derived CCL5 in the cognitive function of APP/PS1 mice, we performed a MWM test on the treated mice. As shown in FIG. 7a, the APP/PS1 mice (injected with PBS) showed significant memory deficits compared with WT mice. Notably, we found that APP/PS1 mice treated with BM-MSCs performed significantly better on the MWM test than PBS-treated counterparts. However, mice treated with BM-MSCs transduced with CCL5 siRNA did not show improved memory function (p>0.05, vs AD/PBS, FIG. 7a).

FIG. 7b shows examples of the swimming traces in each mouse group analyzed by the MWM task on day 10. In the probe trial, APP/PS1 mice treated with CCL5 knockdown BM-MSCs showed a partial but significant decrease of crossing platform number compared to BM-MSC treated mice (p=0.048, vs AD/BM-MSCs, FIG. 7c). The time spent in the target quadrant did not differ among the groups (FIG. 7d).

As set forth above, according to exemplary embodiments of the invention, any one or more active ingredients selected, from the group consisting of a chemoattractant CCL5, a CCL5 expression regulator, and a CCL5 activator are effective in a prevention or treatment of neurodegenerative, such as dementia by recovering damaged memory power. Accordingly, such an active ingredient can be useful as a pharmaceutical composition or a food composition for preventing or treating neurodegenerative diseases.

While the present invention has been shown and described in connection with the exemplary embodiments, it will be apparent to those skilled in the art that modifications and variations can be made without departing from the spirit and scope of the invention as defined by the appended claims.

What is claimed is:

1. A composition for preventing or treating neurodegenerative diseases, comprising one or two or more selected from the group consisting of a chemoattractant CCL5, a CCL5 expression regulator, and a CCL5 activator as an active ingredient.

2. The composition according to claim 1, wherein the CCL5 activator is amyloid β.

3. The composition according to claim 1, wherein the neurodegenerative diseases is one or more diseases selected from the group consisting of a stroke, palsy, memory loss, memory damage, dementia, amnesia, Parkinson's disease, Alzheimer's disease, Pick's disease, Creutzfeld-Kacob disease, Huntington's disease, and Lou Gehrig's disease.

4. A pharmaceutical composition for preventing or treating neurodegenerative diseases, comprising one or two or more selected from the group consisting of a chemoattractant CCL5, a CCL5 expression regulator, and a CCL5 activator as an active ingredient.

5. The pharmaceutical composition according to claim 4, wherein one or two or more selected from the group consisting of the chemoattractant CCL5, the CCL5 expression regulator, and the CCL5 activator is included in 0.1 parts to 50.0 parts by weight relative to 100 parts by weight of the total pharmaceutical composition in the pharmaceutical composition.

6. A food composition for preventing or treating neurodegenerative diseases, comprising one or two or more selected from the group consisting of a chemoattractant CCL5, a CCL5 expression regulator, and a CCL5 activator as an active ingredient.


SEQUENCE LISTING (26 sequences)

SEQ ID NO: 1 - DNA, 20 bp, Artificial Sequence
  CXCL1 forward primer
cacaaaatgtccaagggaag

SEQ ID NO: 2 - DNA, 20 bp, Artificial Sequence
  CXCL1 reverse primer
gcgaaaagaagtgcagagag

SEQ ID NO: 3 - DNA, 20 bp, Artificial Sequence
  M-CSF forward primer
ttccacctgtctgtcctcat

SEQ ID NO: 4 - DNA, 20 bp, Artificial Sequence
  M-CSF reverse primer
agtctgtcttccacctgctg

SEQ ID NO: 5 - DNA, 18 bp, Artificial Sequence
  MIP-1 beta forward primer
acgggggtcaattctaag

SEQ ID NO: 6 - DNA, 18 bp, Artificial Sequence
  MIP-1 beta reverse primer
gccattcctgactccaca

SEQ ID NO: 7 - DNA, 20 bp, Artificial Sequence
  MIP-2 forward primer
acatctgggcaatggaatta

SEQ ID NO: 8 - DNA, 20 bp, Artificial Sequence
  MIP-2 reverse primer
tgaacaaaggcaaggctaac

SEQ ID NO: 9 - DNA, 20 bp, Artificial Sequence
  CCL5 forward primer
aagcaatgacagggaagcta

SEQ ID NO: 10 - DNA, 20 bp, Artificial Sequence
  CCL5 reverse primer
caatcttgcagtcgtgtttg

SEQ ID NO: 11 - DNA, 22 bp, Artificial Sequence
  IDE forward primer
gaagacaaacgggaataccgtg

SEQ ID NO: 12 - DNA, 20 bp, Artificial Sequence
  IDE reverse primer
ccgctgaggacttgtctgtg

SEQ ID NO: 13 - DNA, 20 bp, Artificial Sequence
  NEP forward primer
gaaattcagccaaagcaagc

SEQ ID NO: 14 - DNA, 20 bp, Artificial Sequence
  NEP reverse primer
gatttcggcctgaggaataa

SEQ ID NO: 15 - DNA, 20 bp, Artificial Sequence
  MMP9 forward primer
gccatgcactgggcttagat

SEQ ID NO: 16 - DNA, 23 bp, Artificial Sequence
  MMP9 reverse primer
tctttattcagagggaagccctc

SEQ ID NO: 17 - DNA, 20 bp, Artificial Sequence
  TNF-alpha forward primer
gctccagtgaattcggaaag

SEQ ID NO: 18 - DNA, 20 bp, Artificial Sequence
  TNF-alpha reverse primer
gattatggctcagggtccaa

SEQ ID NO: 19 - DNA, 20 bp, Artificial Sequence
  IL-1 beta forward primer
cccaagcaatacccaaagaa

SEQ ID NO: 20 - DNA, 20 bp, Artificial Sequence
  IL-1 beta reverse primer
gcttgtgctctgcttgtgag

SEQ ID NO: 21 - DNA, 20 bp, Artificial Sequence
  IL-4 forward primer
atccatttgcatgatgctct

SEQ ID NO: 22 - DNA, 20 bp, Artificial Sequence
  IL-4 reverse primer
gagctgcagagactctttcg

SEQ ID NO: 23 - DNA, 20 bp, Artificial Sequence
  YM-1 forward primer
agagcaagaaacaagcatgg

SEQ ID NO: 24 - DNA, 20 bp, Artificial Sequence
  YM-1 reverse primer
ctgtaccagctgggaagaaa

SEQ ID NO: 25 - DNA, 21 bp, Artificial Sequence
  GAPDH forward primer
ttgctgttgaagtcgcaggag

SEQ ID NO: 26 - DNA, 20 bp, Artificial Sequence
  GAPDH reverse primer
tgtgtccgtcgtggatctga